(19) United States
(12) Patent Application Publication
(10) Pub. No.: US 2024/0153645 A1
(43) Pub. Date: May 9, 2024

(54) DETECTING INFECTION SOURCES

(71) Applicant: Kyndryl, Inc., New York, NY (US)

(72) Inventors: Seng Chai Gan, Ashburn, VA (US); Shikhar Kwatra, SAN JOSE, CA (US); Geeth Ranmal de Mel, Warrington (GB); Lan Ngoc Hoang, Lymm (GB)

(21) Appl. No.: 18/052,228

(22) Filed: Nov. 3, 2022

(51) Int. Cl. G16H 50/70; G16H 40/67; G16H 50/80; G16H 70/60

(52) U.S. Cl. CPC G16H 50/70; G16H 40/67; G16H 50/80; G16H 70/60; G16H 50/30

(57) ABSTRACT

A method, computer program product, and system include a processor(s) obtains various data relevant to one or more infected individuals (e.g., location data and infection data. The processor(s) identifies relationships and physical proximity between people comprising the one or more infected individuals and additional individuals, based on the various data. The processor(s) generates, based on the various data, a geofence. The processor(s) utilizes the geofence, the relationships, the physical proximity, and the various data, to predict that a portion of the additional individuals are more likely than not to be infected with the infection. The processor(s) generates a scoring model to identify a source of the infection. The processor(s) applies the scoring model to a group consisting of the one or more individuals, the portion of the additional individuals, and the physical locations, to identify the source of the infection (i.e., a person or physical locations).

### BACKGROUND

In an epidemiological investigation, scientists seek a (human or other animal) source and/or a place where an infection started. A first infected person sought is referred to as “patient zero.” Detecting a source (geographic and personal) of a disease or infection enables healthcare professionals to mitigate the spread of the disease or infection. Identifying a source enables these professionals to detect the individuals affected we well as hotspots for contracting the infection or disease and based on these identifications, take actions to proactively prevent further spread. One proactive action is to treat individuals considered close contacts of the infected and/or those individuals who have recently visited the identified hotspots with a vaccine and/or with other preventative medical treatments.

### SUMMARY

Shortcomings of the prior art are overcome, and additional advantages are provided through the provision of a method for identifying a source of an infection. The method includes, for instance: obtaining, by one or more processors, various data relevant to one or more infected individuals, the various data comprising location data and infection data; identifying, by the one or more processors, relationships and physical proximity between people comprising the one or more infected individuals and additional individuals, based on the various data; generating, by the one or more processors, based on the various data, a geofence; utilizing, by the one or more processors, the geofence, the relationships, the physical proximity, and the various data, to predict that a portion of the additional individuals are more likely than not to be infected with the infection; generating, by the one or more processors, based on the relationships and physical proximity, a scoring model, wherein the scoring model identifies a source of the infection; and applying, by the one or more processors, the scoring model to a group consisting of the one or more individuals, the portion of the additional individuals, and the physical locations, to identify the source of the infection, wherein the source of the infection comprises at least one person from the group or at least one location from the physical locations.

Shortcomings of the prior art are overcome, and additional advantages are provided through the provision of a computer program product for identifying a source of an infection. The computer program product comprises a storage medium readable by a one or more processors and storing instructions for execution by the one or more processors for performing a method. The method includes, for instance: obtaining, by the one or more processors, various data relevant to one or more infected individuals, the various data comprising location data and infection data; identifying, by the one or more processors, relationships and physical proximity between people comprising the one or more infected individuals and additional individuals, based on the various data; generating, by the one or more processors, based on the various data, a geofence; utilizing, by the one or more processors, the geofence, the relationships, the physical proximity, and the various data, to predict that a portion of the additional individuals are more likely than not to be infected with the infection; generating, by the one or more processors, based on the relationships and physical proximity, a scoring model, wherein the scoring model identifies a source of the infection; and applying, by the one or more processors, the scoring model to a group consisting of the one or more individuals, the portion of the additional individuals, and the physical locations, to identify the source of the infection, wherein the source of the infection comprises at least one person from the group or at least one location from the physical locations.

Shortcomings of the prior art are overcome, and additional advantages are provided through the provision of a system for identifying a source of an infection. The system comprises a memory; one or more processors in communication with the memory; program instructions executable by the one or more processors to perform a method. The method can include: obtaining, by the one or more processors, various data relevant to one or more infected individuals, the various data comprising location data and infection data; identifying, by the one or more processors, relationships and physical proximity between people comprising the one or more infected individuals and additional individuals, based on the various data; generating, by the one or more processors, based on the various data, a geofence; utilizing, by the one or more processors, the geofence, the relationships, the physical proximity, and the various data, to predict that a portion of the additional individuals are more likely than not to be infected with the infection; generating, by the one or more processors, based on the relationships and physical proximity, a scoring model, wherein the scoring model identifies a source of the infection; and applying, by the one or more processors, the scoring model to a group consisting of the one or more individuals, the portion of the additional individuals, and the physical locations, to identify the source of the infection, wherein the source of the infection comprises at least one person from the group or at least one location from the physical locations.

Methods, computer program products, and systems relating to one or more aspects are also described and claimed herein. Further, services relating to one or more aspects are also described and may be claimed herein.

Additional features are realized through the techniques described herein. Other embodiments and aspects are described in detail herein and are considered a part of the claimed aspects.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 is a workflow that illustrates various aspects of some embodiments of the present invention;

FIG. 2 is an example of a computer system into which aspects of the present invention can be implemented;

FIG. 2 provides a general overview of how the program code in some examples can identify a location as a source of an infection;

FIG. 3 is a non-limiting example of an architectural flow of determinations made by the program code to predict a likelihood of infections for individuals in contact with an infected person and based on these predictions, to identify a given individual as a source of the infection as well as predicting risks for the other individuals over time;

FIG. 4 is a system diagram that graphically illustrates various aspects of the architectural flow of FIG. 3;

FIG. 5 is a workflow that illustrates various aspects of some examples discussed herein;

FIG. 6 is a workflow that illustrates various aspects of some examples discussed herein;

FIG. 7 depicts on embodiment of a computing node that can be utilized in a cloud computing environment;

FIG. 8 depicts a cloud computing environment according to an embodiment of the present invention; and

FIG. 9 depicts abstraction model layers according to an embodiment of the present invention.

### DETAILED DESCRIPTION

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention. As understood by one of skill in the art, the accompanying figures are provided for ease of understanding and illustrate aspects of certain embodiments of the present invention. The invention is not limited to the embodiments depicted in the figures.

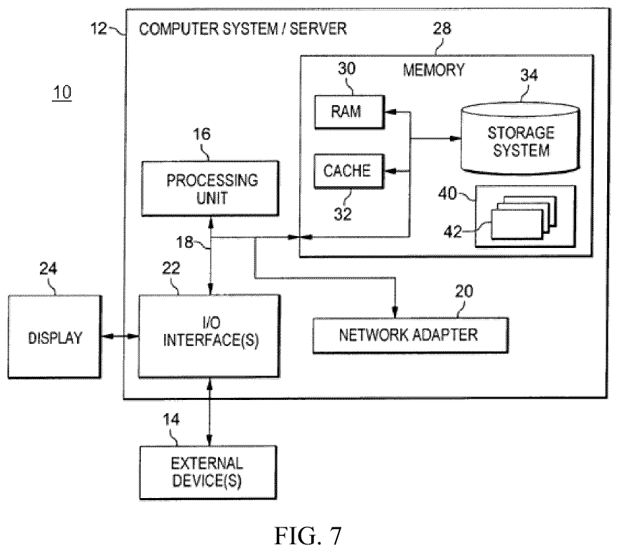

As understood by one of skill in the art, program code, as referred to throughout this application, includes both software and hardware. For example, program code in certain embodiments of the present invention includes fixed function hardware, while other embodiments utilized a software-based implementation of the functionality described. Certain embodiments combine both types of program code. One example of program code, also referred to as one or more programs, is depicted in FIG. 7 as program/utility 40, having a set (at least one) of program modules 42, may be stored in memory 28.

Herein the terms “infection” and “disease” as used interchangeably to represent a transmissible malady, including but not limited to, a bacteria, a virus, etc.

Herein the term “individual” refers to a person or a group of people who has/have provided consent to participate in an implementation of the infection detection techniques described herein. In these examples, the “monitoring” of individuals relates to determining the presence and/or absence of the individuals at public places. The individuals are identified by the program code in the “monitoring” based on unique identifiers, including those associated with personal devices utilized by the individuals. The monitoring described herein does not collect or utilize personally identifiable data (PID) beyond data provided by individuals, to determine presence and/or absence at a location. For example, the examples herein discuss program code identifying relationships and physical proximity of individuals. The information utilized by the program code to identify relationships is provided by the individuals either manually and/or by opting into providing access to the program code. The program code determines that there is a relationship between more than one individual only if all individuals within the relationship have consented to participate in the monitoring. Thus, any data obtained by the program code in the examples below from individuals, whether the entities are labeled “individuals” or “additional individuals”, the use of the term “individual” indicates a person who has consented to participate in the detection of infections by the program code as described herein,

Herein the term “infection data” refers generally to data comprising parameters related to an infection. These event data can include diagnostic data (e.g., information collected and used in the investigation and diagnosis of a disease), target data (e.g., characteristics of a target product that is aimed at a particular disease or diseases), event data (e.g., events that represents an immediate threat to human health and require prompt action), transmission data (e.g., manners in which germs are moved to the susceptible person), and/or infection data (e.g., a probability or risk of an infection in a population).

Herein the term “location data” refers to one or more of knowledge representation data of one or more individuals with a given infection and/or geospatial data related to physical locations proximate to the one or more individuals.

Embodiments of the present invention include systems, computer program products, and computer-implemented methods where program code executing on one or more processors generates and applies a scoring model to identify an individual who is most likely a source of an infection. To generate and apply this scoring model, program code in embodiments of the present invention utilizes geofencing to identify and monitor people who are within a proximity of an infected person at a time of possible infection. In various embodiments of the present invention, program code executing on one or more processors generates a knowledge graph to enable personalized tracking of individuals and based, in part, on the knowledge graph, the program code estimates a likelihood for each individual that this individual is a source for a given infection. The results generated by the program code can be inclusive of risks for further infection.

Embodiments of the present invention include program code that determines routes taken by infections and detects spread of the infection based on convergence by utilizing a combination of geofencing and a social network and utilizing a Bayesian network (BN) taught utilizing a convolutional neural network (CNN) comprising a long short-term memory (LSTM) network. In certain of the examples herein, program code utilizes a BN or a Bayesian inference model (Bayesian models are fundamentally cause-and-effect), to makes inferences (e.g., statistical inferences) to fill in missing data points and generate root cause analyses indicating the location of hotspots for a given infection and/or the identity of individuals who are likely sources of the given infection. CNNs utilize feed-forward artificial neural networks, including in some examples herein, an LSTM, and utilize convolutional layers that apply a convolution operation (a mathematical operation on two functions to produce a third function that expresses how the shape of one is modified by the other) to the input, passing the result to the next layer. The convolution emulates the response of an individual neuron to visual stimuli. Each convolutional neuron processes data only for its receptive field.

Utilizing a CNN reduces the number of free parameters, allowing the network to be deeper with fewer parameters, thus, working with the BN and an LSTM layer to identify a source for a given infection, the CNN can utilize a consistent number of learnable parameters because CNNs fine-tune large amounts of parameters and massive pre-labeled datasets to support a learning process. CNNs are composed by a feature extraction block and a classification block. The feature extraction block receives a grid-like topology input and extracts representative features in a hierarchical manner. The classification block receives the top hierarchical feature and delivers a final matrix of prediction. When an LSTM is combined with a convolutional layer, the forget gate of the LSTM can serve to further streamline data over time.

CNNs resolve the vanishing or exploding gradients problem in training traditional multi-layer neural networks, with many layers, by using backpropagation. Thus, CNNs can be utilized in large-scale recognition systems, giving state-of-the-art results in segmentation, object detection (e.g., detection of a first infected individual) and object retrieval. Utilizing an LSTM network in examples herein enables the program code to make predictions (e.g., determine a path for an infection) based on time series data. CNNs, when applied in pattern and vision recognition, use a weighted combination of each pixel and their filtered neighbors, to train the network. Meanwhile, an LSTM has a chain-like structure of a neural network layers with a forget gate layer to allow selecting relevant information. LSTMs have three layers: an input gate, an output gate, and a forget gate. The forget gate decides how much of the previous data will be forgotten and how much of the previous data will be used in next steps. For example, a result of a forget can be in the range of 0-1 while “0” forgets the previous data, “1” uses the previous data. Forget gate layer can be modelled as in equation 1. The program code utilizes a convolutional LSTM which provides robust in pattern recognition while accounting for changes over time. Thus, embodiments of the present invention combine various advantages and functionalities of both convolutional and LSTM layers in a neural network.

In some examples herein, the CNN, which includes convolutional blocks, utilized by the program code, utilizes maximum pooling (“max pooling”) as its encoding network. Max pooling summarizes the most activated presence of a feature. Pooling layers in a CNN down sample the detection of features in a feature map by summarizing the presence of features in patches of the feature map. As will be discussed herein, embodiments of the present invention utilize this feature of CNNs to generate a heat map to indicate hotspots for a given infection. Generally, convolutional layers in a CNN systematically apply learned filters to inputs to create feature maps that summarize the presence of features in the inputs. A pooling layer is a new layer added after a convolutional layer. An example of an order of layers in a CNN that includes a pooling layer is as follows: 1) input layer) convolutional layer; 3) nonlinearity; and 4) pooling layer. In architectures such as this example, where the pooling layer is added after the convolutional layer, the pooling layer operates upon each feature map separately to create a new set of the same number of pooled feature maps. Applying the pooling layer reduces the size of a feature map, in the case of max pooling, by calculating a maximum value for each patch of the feature map. The pooling layer creates a down sampled or pooled feature maps with a summarized version of the features detected in the input.

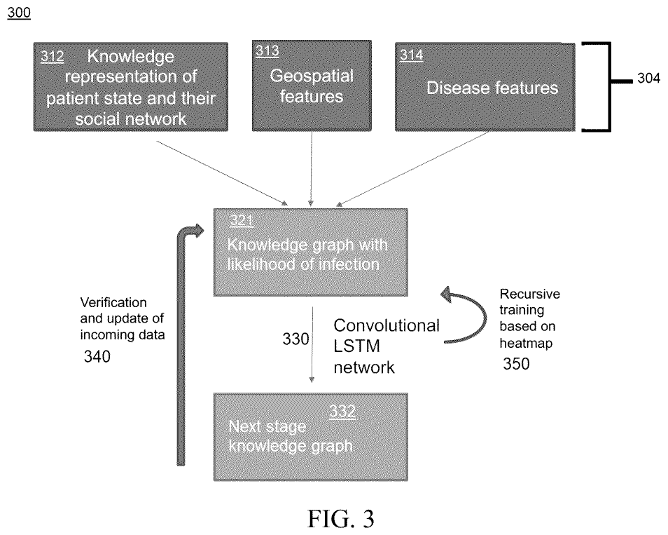

In some embodiments herein, the program code supplements the CNN and LSTM by adding a dense layer. A dense layer is a deeply connected layer (i.e., neurons of the layer are connected to every neuron of its preceding layer) from its preceding layer which changes a dimension of an output by performing matrix vector multiplication. Different layers in a neural network (which can be understood as a deep learning model) provide different utility to the neural network. LSTM layers are utilized mostly in time series analyses (as illustrated in FIG. 3) or in the natural language processing (NLP) problems while convolutional layers generally provide image processing, etc. As discussed herein, the LSTM layers are provided in the examples herein for their timing-related utility. A dense layer, also referred to as a fully connected layer, is used in the final stages of a neural network because it can change dimensionality of an output from a preceding layer so that the model can define a relationship between the values of the data in which the model is working. The dense layer's neuron in a model receives output from every neuron of its preceding layer, where neurons of the dense layer perform matrix-vector multiplication. Thus, the dense layer can normalize the outputs of both convolutional layers and the LSTM to generate a cohesive knowledge graph that provide/predicts a likelihood of infection for an individual and/or a likelihood that the individual is a source of an infection.

In embodiments of the present invention, program code executing on one or more processors: 1) obtains identifications of infected individuals; 2) utilizes a geofence and tracking to identify individuals who were in contact with the infected individuals; and 3) generates and implements a scoring model to identify which individual of the infected individuals is most likely the source of the infection (e.g., patient zero); and 4) automatically (regularly) updates the model based on machine learning from past infections. The updates to the model, through machine learning, enable the program code to narrow a location and a source of each infection.

Embodiments of the present invention provide significantly more than existing approaches to identify an individual who is most likely a source of an infection. Existing approaches rely on either collecting medical samples from individuals and testing those samples or producing reports based on collected data. Examples herein provide significantly more than these approaches at least because they provide predictive information that not only identifies a likely source of an infection but also monitors contacts within a geofence of the infected person at the time of possible infection to determine a scoring model to anticipate a spread of an infection. The program code generates a personalized knowledge graph to track infected individuals and estimate a likelihood of each individual of the individuals being the source of the infection, and determines risks associated with each individual for further infection.

Embodiments of the present invention are inextricably linked to computing and provide a practical application. Program code in embodiments of the present invention not only identifies a source for an infection but can anticipate a route of an infection based on this personalized analysis based on a convergence using a Bayesian network inculcated with a convolutional LSTM framework. Various of these aspects are inextricably tied to computing. Additionally, some embodiments of the present invention utilize fine grain awareness provided by 5G, which can locate individuals with an accuracy of two meters, to locate individuals and therefore, determine contacts with various infected individuals and/or set the bounds of geofences described herein. Some embodiments herein include program code that utilizes location awareness provided by antenna arrays with beamforming capabilities, which are part of a cellular network, which enable the program code to locate individuals with a single base station as a reference point. Embodiments described herein are directed to the practical application of providing healthcare resources to areas and people in need and identifying these areas and people in advance of infections to enable efficient and optimized distributions of medical resources.

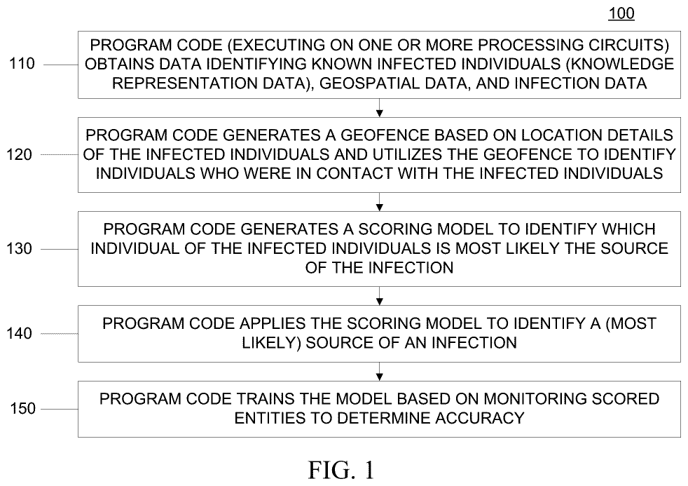

FIG. 1 illustrates a workflow 100 of these general aspects. The program code (executing on one or more processing circuits) in some examples obtains data identifying known infected individuals (e.g., knowledge representation data), infection (or disease) data, and geospatial data (110). These data, which is discussed in greater detail herein, including in FIG. 3, can include, but are not limited to, data representing both a state of a patient (health, medical concerns, levels of activity, etc.), as well as social network information, which can be obtained by the program code based on interactions of the individuals with social media sites, check-in sites, and surveillance of the individual based on devices, including Internet of Things (IoT) devices, carried by the user and proximate to the user. The infection data includes various parameters related to the transmissibility of the infection discussed in more detail herein.

The program code generates a geofence based on the data and utilizes the geofence to identify additional individuals who were in contact with the infected individuals where the contact meets a threshold for likely transmission of the infection to the additional individual (120). The program code utilizes the geospatial data to determine the location details of the individual. The program code selects a time window for when these location details are relevant based on the infection data. For example, the locations of an infected individual during the time when this individual can transmit the infection are relevant to the program code when the program code generates the geofence. The individuals identified by the program code as possibly infected are those within the geofence boundaries at a time when based on the infection parameters, these people are likely to have contracted the infection. Because the program code obtains infection data (related to the infection) as well as the proximity data, the program code can determine that a given individual was more likely than not to be infected based on a contact with an infected individual. For example, the program code can obtain data indicating that a given infection is communicative through the air provided that an infected person is within 5 meters of another individual for more than five minutes. The program code can determine that this type of contact occurred and thus, the individual who was in contact with the infected person would be more likely than not to be infected (e.g., the probability of infection would exceed a threshold). Provided that a contact meets the criteria for transmission for a given infection, as defined by the infection data, the program code can predict a transmission of that infection.

With these additional identifications, the program code generates a scoring model to identify which individual of the infected individuals is most likely the source of the infection (e.g., patient zero) (130). FIG. 3 demonstrates how program code can utilize a Bayesian network to identify a likelihood that each individual is the source of the infection (e.g., patient zero). The Bayesian network enables the program code to infer, based on the data obtained, as well by filling in missing data points, to generate root cause analyses that indicate a likelihood that each infected individual is a source and/or that each location is a hotspot. In some examples, the program code quantifies likelihood that a given individual is patient zero based on a threshold number of connections between that individual and other infected individuals and/or based on the presence and/or absence of that individual at hotspots during a window of time where transmission would occur, based the parameters of the infection. Thus, the program code utilizes a Bayesian network to analyze relationships between the initial people infected, the people adjudged infected based on contacts (as determined with the help of the geofence), the geospatial data, and the data identifying one or more individuals (e.g., a knowledge representation of a patient state and the patient's social network), to generate a scoring model to identify a source of the infection.

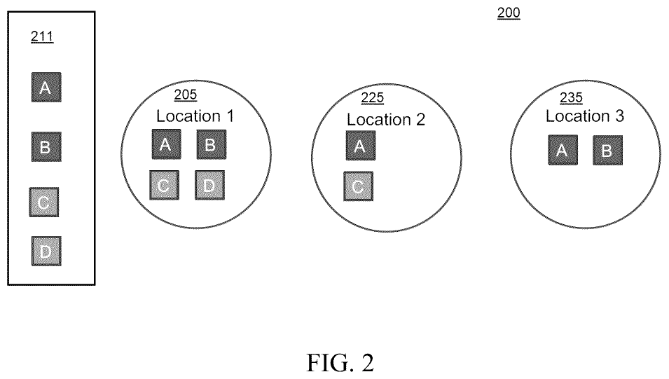

The program code applies or the scoring model to identify a (e.g., most likely) source of an infection (140). The source identified by the program code can be a location and/or an individual. FIG. 2 provides a general overview of how the program code in some examples can identify a location as a source of an infection.

Returning to FIG. 1, the program code trains the model based on monitoring scored entities to determine accuracy (150). In some examples, the program code does not monitor scored entities, but, rather, obtains the actual results based on reporting and data entry, including self-reporting. In some example, the program code can interface with a medical records system (provided appropriate permissions are in place) to obtain the actual results. The program code can compare these actual results to the forecasted results of the scoring model.

To identify a most likely candidate as a source of an infection, program code in some examples herein can monitor individuals within a geofence of the infected person at the time of possible infection to generate the scoring model. Program code in embodiments of the present invention can identify source infections and identify individuals who have a probability of being infected (e.g., within a pre-defined range) by navigating in two directions (e.g., up, and down) through a tree generated by the program code to represent an infection pattern. A non-limiting practical application of embodiments of the present invention is that they enable deployment of medical interventions (medicines, vaccines, etc.) to individuals the program code identifies as high risk, or who were in a geographic area that the program code identifies as high risk. Thus, embodiments of the present invention assist in focusing the use of potentially limited medical resources to optimize efficacy in mitigating the spread of an infection (e.g., virus, bacteria, illness, etc.).

In embodiments of the present invention, program code utilizes a geofence and tracking to identify individuals who were in contact with the infected individuals. The program code utilizes the geofence to detect all other people and objects that come into contact and a length of the contacts (e.g., FIG. 1, 120). When an approximate time of an infection is known (based on incubation period, expression of symptoms, etc.), the program code can trace the route of the infection using individuals who are infected and not infected. The program code generates and utilizes a geofence (e.g., FIG. 1, 120) to detect other people and objects that come into contact and length of the contacts with a given infected individual. In addition to utilizing a geofence, which assists the program code in determining physical proximity, in embodiments of the present invention, the program code utilizes data available on social media, including social network information (e.g., friends, neighbors, family, co-workers) and makes proximity inferences to detect possible physical contacts to supplement the actual location/route information. Thus, when the program code scores an individual as less or more likely to be a source of an infection (e.g., FIG. 1, 130), the program code utilizes, in some examples, both the proximity of the social network, of a surveillance/monitoring network (e.g., determining the location of the individual utilizing devices including but not limited to Internet of Things devices), and a length of the contact with an infected individual.

As will be discussed herein, in addition to obtaining information related to individuals and geospatial data, program code in embodiments of the present invention also obtains parameters related to the disease. There are various configurable parameters in various examples herein, including but not limited to, configurations related to the disease or infection that is traced by the program code and/or configurations related to the person(s) of interest used by the program code to trace the spread of the disease as well as to identify the source of the disease. Once the program code determines a source for an infection (e.g., patient zero), active measures can be taken, including but not limited to instituting a quarantine for the source.

Configurable parameters related to the disease can include but are not limited to: incubation period (i.e., time it takes for the symptom to present), infection radius (i.e., distance for an infected person to infect others, including an indoor and/or an outdoor radius, which will differ), infection vector (i.e., how the disease can be transmitted from one individual to another, including but not limited to air-borne, water-borne, mosquitos, rodent, etc.), personal traits associated with a sensitivity to contracting the disease (i.e., a phenotype associated with an individual who could be more likely to contract the disease, such as someone with an existing underlying condition or co-morbidity), degree of contagion, time window for the disease to resolve or terminate the subject, and/or management or mitigation options (treatments, vaccines, therapies, etc.).

A knowledge representation of an individual (e.g., FIG. 1, 110, FIG. 3, 312) can include various configurable parameters. Configurable parameters related to the person(s) of interest can include but are not limited to: whether the individual is symptomatic, the location of the individual during a time-period of interest, and/or data sources with information about the individual which enables the program code to tune the location of the individual and predict close contacts of the individual during a time-period at which the individual could potentially spread the disease. There are various sources from which data can be obtained by the program code to determine contacts of individuals of interest, including but not limited to soft sources (social, surveillance) which can be utilized by the program code to localize a relationship of a person-of-interest. The program code can make proximity inferences based on check-ins on social networks. The program code can also utilize these check-ins to geofence potentially infected individuals. The program code can also perform edge processing with respect to proximity models to infer closeness to a person-of-interest using surveillance networks. Social networks also provide data utilized by the program code by way of relationships revealed by user profiles of potentially infected individuals. Program code can prune potentially infected persons by focusing on the social network relationships (e.g., family member, friend, neighbor, co-worker, etc.). Depending on technology utilized individuals and the permissions they grant to their devices, or had granted to their devices, the program code may be able to obtain route and/or mobility information related to the individuals. Based on this tracking information, the program code can determine and/or approximate movement zones. Additionally, the program code can determine if these transportation routes were potentially shared (causing possible exposure to others). Shared routes can include, but are not limited to places where space is shared by travelers, for example in buses, trains, restaurants, etc.

As noted above, FIG. 2 provides a general overview 200 of how location data related to individuals can assist program code in determining a physical source of an infection. This example is heavily simplified for illustrative purposes. The example 200 utilizes four individuals 211, A, B, C, and D. Of the individuals 211, A and B are infected while C and D are not infected. During the time when transmission/infection can occur, the individuals 211 were at various locations, 205, 225, 235, which are also labeled Location 1, Location 2, and Location 3. All individuals 211 were at Location 1 205. A and C were at Location 2 225, and A and B were at Location 3 235. The only location where both infected people were present without any non-infected people was Location 3 235. Thus, Location 3 235 is likely the source of the infection.

FIG. 3 is a non-limiting example of an architectural flow 300 of determinations made by the program code to predict a likelihood of infections for individuals in contact with an infected person and based on these predictions, to identify a given individual as a source of the infection as well as predicting risks for the other individuals over time. This likelihood of infection factors into the program code determining/verifying a source of the infection. FIG. 3 can be understood in the context of the example provided in FIG. 2 as well as certain aspects illustrated in FIG. 1.

Returning to FIG. 1, once the program code obtains data identifying an infected person (110), and/or determines who is infected, the program code can then develop a scoring model (130) and apply the scoring model (140). The information identifying the infected person can also include the approximate time of the infection (e.g., 3 or 4 days before the program code generates the scoring model and the program code applies the scoring model). Returning to FIG. 2, if one assumes that the infected person of the individuals 211 is A, the program code can develop a scoring model (FIG. 1, 130), based on one or more of the following factors: 1) a length of time in a geofence between A and another person (e.g., B); 2) a social/geospatial relationship between the person A and B; 3) time/frequencies of interaction between A and B.

As illustrated in FIG. 3, the program code obtains, as inputs 304, a knowledge representation of patient state and the social network of the patient 312 (which can include the aforementioned configurable parameters), geospatial features 313, and disease features 314 (e.g., configurable parameters including but not limited to, incubation period, transmissibility, etc.). Based on these inputs 304, the program code generates a knowledge graph that represents likelihood of infection 321. The program code can also utilize the knowledge graph to determine a likelihood of infection of whether a given individual is a source of the infection. As discussed above, when the program code utilizes a utilizes a Bayesian network to identify causes and effects, the program code can navigate both ways (e.g., up, and down causation models) and thus, the program code can utilize the knowledge graphs moving forward in time for prediction and backward in time for source attribution (i.e., to identify the source)).

The knowledge representation of patient (individual) state and the social network of the patient 312 comprises attributes relevant to the individual. These attributes include, broadly, where the individual has been during a relevant time-period (as informed by the disease features 314) and personal attributes relevant to the individual that impact the individual's probability of transmitting the infection to others. The program code generates this knowledge graph 321 based on the inputs (attributes) 304, which can include, but are not limited to, coordinates at which the individual (e.g., A), was present. The program code can obtain these coordinates based on information provided by the individual, including when the individual uses a personal computing device to check in at venues via an interface to a social network. The knowledge representation data 312 can also include proximity information from both social and surveillance networks. Surveillance networks can include information from cell towers regarding connections made by a device utilized by the user, sensors, sensors related to traffic management (EZ Pass, etc.). Certain personal preferences from social networks profiles of the user can also be used by the program code to assist the program code in determining transmission risk, especially attributes which would expedite the spread of the disease, including but not limited to preferences/beliefs of the user adverse to vaccination. These knowledge representations 312 obtained by the program code can include, but are not limited to, text and images on an individual's social media profile. Additional data can include infection state of the individual and the individual's (physical) radius of interaction.

The program code, in generating the knowledge graph, can determine which knowledge representations 312 are relevant based on also obtaining geospatial features 313 and disease features 314. The relevance and types of disease features 314 was discussed earlier but when plotting transmission of an infection, attributes of the disease related to timing are relevant to the program code, including but not limited to, incubation period, transmissibility window, etc. The geospatial features of the 313 of a physical environment of the individual, which would include, for example, referring to FIG. 2, Location 1 205, Location 2 225, and Location 3 235, are utilized by the program code to provide results to a user, including but not limited to, a heatmap, to indicate, for example, that Location 3 235 is a hotspot or source of infection.

The program code utilizes the inputs 304 to generate a knowledge graph 321. Each knowledge graph generated by the program code is specific to an individual (i.e., personalized). In some examples, the program code generates the knowledge graph by utilizing recursive likelihood estimation (e.g., P(B|A)) based on combination of risk factors, such as via a Bayesian network and conditional calculation (to determine likelihood of being infected). P(B|A) represents a probability of being infected (also referred to herein as likelihood of being infected), specifically, in this example, the probability of being infected with B given the contextual information A, present subjective knowledge. A knowledge graph 312 is specific to a given user (e.g., individual A). When two or more knowledge graphs overlap, subject to a pre-defined threshold, possible disease profiles convergence is exhibited. A given disease threshold is subject to the disease, as discussed herein and the configurations or parameters related to the disease. As the contextual information changes (e.g., knowledge representations 312, geospatial features 313, disease features 314), the program code updates the knowledge graph 312. The program code can obtain updates continuously, periodically, etc. The program code can utilize the early data/back casting results to trace an origin of a disease. Based on identifying an origin, preventive measures can be taken to mitigate further spread. The program code can also identify current hotspots and next vulnerable individuals. The program code can superimpose the knowledge graph 321 on a geospatial map to generate a heat map of disease risks and/or a timeline of potential disease propagation. The program code utilizes the knowledge graphs of the individuals, which indicate the likelihood of infection, to identify a likely source of the infection.

The program code generates the heat map or timeline of potential risks based on classifying hotspots and evolving this disease heat map over time. In some examples, the program code utilizes a convolutional LSTM to classify hotspots and evolve the heat map over time (330). The convolutional LSTM provides robust pattern recognition by preserving the knowledge/geospatial information and provides predictions based on how the knowledge graphs change over time. The program code utilizes the inputs of the knowledge graphs and/or the knowledge graphs themselves as inputs into the LSTM in a past time step to generate a new heatmap to reflect how the disease spreads and changes over the patients and their social networks. In some embodiments of the present invention, the neural network comprising the LSTM is executed on a technical infrastructure comprising one or more of a mesh, grid, or graph structure. This convolutional network can include multiple convolutional blocks trained to encode risk classification from input features (e.g., knowledge representations 312, geospatial features 313, disease features 314). The LSTM layer then predicts how the risk classification changes across time, based on the input features, over time, which the program code verifies. The program code updates the knowledge graphs 321 for each individual based on actual and/or predicted changes to the risk classifications.

As illustrated in FIG. 3, the program code verifies inputs of incoming data (as inputs continue after the program code generates the initial knowledge graph 321) (340). The program code tunes the knowledge graph 321, over time, utilizing the convolutional LSTM, thus generating a new (e.g., next) stage knowledge graph 332. The program code recursively generates the knowledge graph such that it increases in accuracy over time (350). Utilization of the convolutional LSTM by the program code preserves the knowledge/geospatial and enables the program code to predict how the knowledge graphs change over time, moving (e.g., cyclically) from the knowledge graph 321 to the next stage knowledge graph 332. Thus, the evolving knowledge graphs 321 332 serve as inputs in a past time step such that the program code can generate a new heatmap, as inputs change over time, to reflect how the disease spread and changes (over the individuals and over the social networks of the individuals).

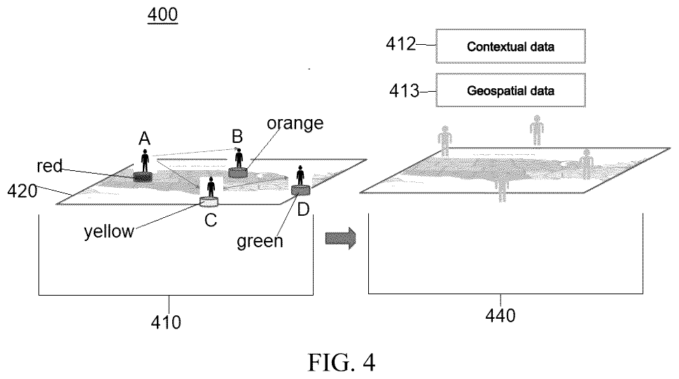

FIG. 4 is a system diagram 400 that graphically illustrates various aspects also illustrated in FIG. 3. In FIG. 4, diagram 410 illustrates an analysis of relationships and connections performed by the program code utilizing a Bayesian network. In this example, colors indicate a likelihood being an infection source. Individual A is on a red indicator, individual B is standing on an orange indicator, individual C is on a yellow platform, and individual D is on a green platform. The red platform indicates that individual A is more likely to be the infection source. Individual B, on an orange indicator so this individual is less likely that individual A to be the infection source. Individual C (yellow), is less likely than B of being the source and individual D (green) is least likely of being the infection source. Individual D has the lowest risk of being the infection source as this individual is mostly outside of a geofence 420 generated by the program code. To generate a knowledge graph (e.g., FIG. 3, 321, 332), the program code obtains contextual data 412 (e.g., FIG. 312, 314) and geospatial data 413 (e.g., FIG. 3, 313). The program code generates the knowledge graph by performing an agent-based simulation and/or utilizes a convolutional LSTM to generate a graph or other representation and clusters and tags the high-risk portions of the graph (taking into account the likelihood that each individual is the source as well geographic and individual contextual data). The program code provides, as inputs to the convolutional LSTM, the likelihood of each individual being an infection source, based on the Bayesian network and the geofencing, and/or contextual data related to individual risks for further infection. The program code generates a heat map 440 representing risk predictions made by the program code. In the heat map 440, the program code has utilized the convolutional network for graph clustering and tagging high risk parts of the graph.

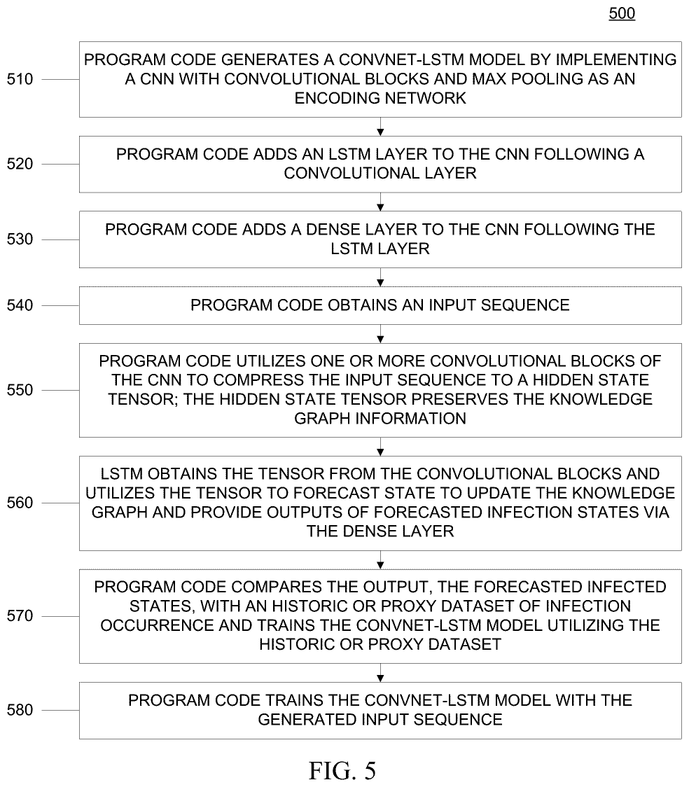

FIG. 5 is a workflow 500 that includes various aspects of some examples describes herein including both the configuration of aspects of a technical environment which executes aspects of forecasting infection sources and associated future risks related to the infection. Initially, the program code executing on one or more processors constructs a Convnet-LSTM model (convolutional network with LSTM model, also referred to as a scoring model in FIG. 1) for predicting sources of an infection and forecasting future infections. To this end, the program code implements a CNN with convolutional blocks and max pooling as an encoding network (510). The program code adds an LSTM layer to the CNN after a convolutional layer (520). The program code adds a dense layer to the CNN after the LSTM layer (530). The dense layer can consolidate the output of the convolutional and LSTM layers.

The program code obtains an input sequence (540). The input sequence includes knowledge graphs, generated, and updated over time, (e.g., generated by the program code as illustrated in FIG. 3), and additional contextual and geospatial data, including but not limited to, features associated with infection status, locations of infections, distance to nearest sources, etc. The program code can utilize the knowledge graphs moving forward in time for prediction and backward in time for source attribution.

The program code utilizes one or more convolutional blocks of the CNN to compress the input sequence to a hidden state tensor; the hidden state tensor preserves the knowledge graph information (550). A tensor is a mathematical object analogous to but more general than a vector, represented by an array of components that are functions of the coordinates of a space. A hidden state result in a neural network refers to an intermediate representations of input within a neural system. Thus, this tensor is an intermediate result. Ultimately, the neural network will re-represent an input in a specific way so that the system can produce some target output. Each layer within a neural network perceives input according to the specifics of its nodes, so each layer of the neural network produces unique snapshots of the data being processed. Hidden states are intermediate snapshots of the original input data, transformed according to a given layer's nodes and neural weighting. The LSTM obtains the tensor from the convolutional blocks and utilizes the tensor to forecast state to update the knowledge graph and provide outputs of forecasted infection states via the dense layer (560). These forecasted infection states can also be understood as changes in the risk classifications of the individuals.

The Convnet-LSTM model is a scoring model that determines the probability of likelihood that individuals and places are sources of an infection. (In some embodiments, the scoring model can determine the likelihood an individual is infected or will become infected because of the flexibility of the knowledge graphs.) As illustrated in FIG. 4, the degrees of probability related to being an infection source can be represented by colors and/or other predefined values that represent different threshold probabilities. For example, a given probability range can represent a high or medium or loss risk. The program code can provide the output to a user as a heatmap that indicates probabilities of various areas and individuals being sources of an infection and therefore, enables users to avoid areas and individuals, and enables medical supply distributors to target areas and individuals for treatment. As aforementioned, the program code can look both back and forward (up and down), identifying both sources as well as forecasting patterns for new infections. Thus, not only can the program code identify sources of an infection, but the program code can also forecast the progression of the infection.

In this example, the forecast provided by the program code, indicating the probabilities related to individuals, can be represented by the pseudocode below, where ConvNet represents the convolutional network.

Forecast=ConvNet−LSTM(knowledge graph)

The loss in this example can be represented by the pseudocode below.

Loss=cross-entropy between Forecast and infection

Returning to FIG. 5, the program code trains the Convnet-LSTM model, the scoring model, to increase its accuracy over time and specifically to minimize the cross-entropy loss, with early-stopping. To this end, the program code compares the output, the forecasted infected states, with an historic or proxy dataset of infection occurrence and generates an input sequence to train the Convnet-LSTM model utilizing the historic or proxy dataset (570), provided that the historic or proxy dataset is of an acceptable common ratio between training and testing (e.g., 80:20, but can vary based on available data). The program code trains the Convnet-LSTM model with the generated input sequence (580). When training the Convnet-LSTM model (as well as when utilizing the model), the input sequence provided to the program code the input sequence varies depending on the spreading speed and infection rate of the disease.

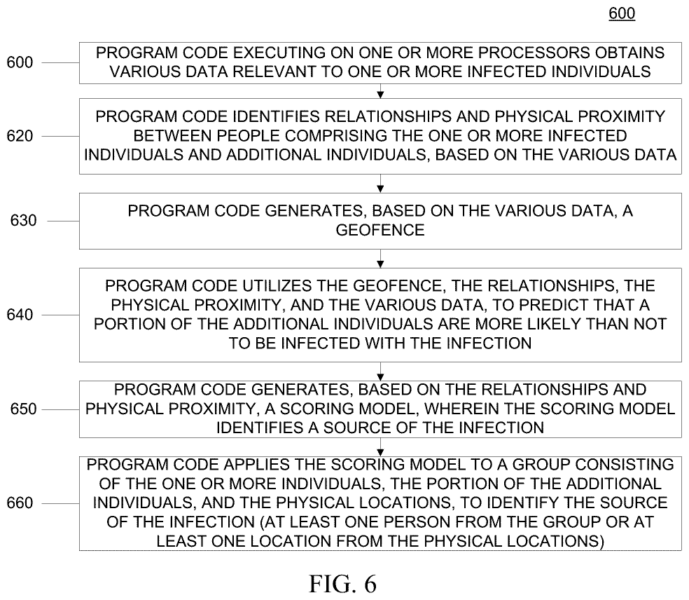

FIG. 6 is a workflow 600 that illustrates various aspects of some embodiments of the present invention. As noted above, all individual whether infected or noted as the additional individuals have consented to participation in the service and/or process described. As illustrated in FIG. 6, the program code executing on one or more processors obtains various data relevant to one or more infected individuals (610). These data can include location data and infection data. The program code identifies relationships and physical proximity between people comprising the one or more infected individuals and additional individuals, based on the various data (620). The program code generates, based on the various data, a geofence (630). The program code utilizes the geofence, the relationships, the physical proximity, and the various data, to predict that a portion of the additional individuals are more likely than not to be infected with the infection (640). The program code generates, based on the relationships and physical proximity, a scoring model, wherein the scoring model identifies a source of the infection (650). The program code applies the scoring model to a group consisting of the one or more individuals, the portion of the additional individuals, and the physical locations, to identify the source of the infection, wherein the source of the infection comprises at least one person from the group or at least one location from the physical locations (660). The individual and/or location that the program code utilizes the scoring model to identify is a most likely source of infection. As explained herein, the scoring model can score these various entities and the scores indicate likelihoods that each entity it scores is the source.

Embodiments of the present invention include a computer-implemented method, a computer program product, and a computer system where program code executing on one or more processors obtains various data relevant to one or more infected individuals, the various data comprising location data and infection data. The program code identifies relationships and physical proximity between people comprising the one or more infected individuals and additional individuals, based on the various data. The program code generates, based on the various data, a geofence. The program code utilizes the geofence, the relationships, the physical proximity, and the various data, to predict that a portion of the additional individuals are more likely than not to be infected with the infection. The program code generates, based on the relationships and physical proximity, a scoring model, wherein the scoring model assigns scores to entities comprising a group consisting of the one or more individuals, the portion of the additional individuals, and the physical locations to identify a source of the infection. The program code applies the scoring model to the group, to identify the source of the infection, wherein the source of the infection comprises at least one person from the group or at least one location from the physical locations.

In some examples, identifying the relationships and the physical proximity comprises the program code utilizing a Bayesian network.

In some examples, the program code utilizes the geofence, the relationships, the physical proximity, and the various data, to predict the portion by: determining if an individual of the additional individuals is part of the portion by determining if the individual had a contact with at least one of the one or more infected individuals (the contact is more likely than not to transmit the infection, based on at least a portion of the various data).

In some examples, the various data further comprise location information for at least one infected individual from a social network or a surveillance network.

In some examples, based on applying the scoring model, the program code generates a knowledge graph indicating a likelihood at least one person from the group entity is the source of the infection.

In some examples, the various data comprise geospatial data related to physical locations proximate to the one or more infected individuals and the geospatial data comprise a geospatial map. The program code can generate a heat map of infection risks based on superimposing the knowledge graph on the geospatial map.

In some examples, the program code generates, based on applying the scoring model, a timeline of propagation for the infection.

In some examples, the program code provides the knowledge graph to a neural network comprising a convolutional layer and a long short-term memory layer. The convolutional layer can encode risk classifications based on a portion of the various data, and the long short-term memory can layer predict changes in the risk classifications over time. The program code updates the knowledge graph based on the risk classifications.

In some examples, based on applying the scoring model, the program code generates a knowledge graph indicating a likelihood at least one individual of the portion is the source of the infection. The program code monitors the at least one individual to determine actual results regarding whether the at least one individual is infected with the infection. The program code compares the actual results with the knowledge graph to identify discrepancies. The program code updates the scoring model to address the discrepancies.

In some examples, the program code compares results of applying the scoring model with a dataset selected from the group consisting of: an historic dataset of infection occurrence or a proxy dataset of infection occurrence. Based on the comparing, the program code generates an input sequence to train the scoring model. The program code provides the input sequence to a neural network. The program code updates the scoring model based on output from the neural network generated from the input sequence.

In some examples, the program code generates the scoring model by implementing a neural network. The program code can implement the neural network by implementing the convolutional layer (e.g., at least one convolutional block). In some examples, an encoding network of the convolutional layer comprises maximum pooling; the convolutional layer compresses the knowledge graph for each entity into a hidden state tensor. The program code adds the long short-term memory layer to obtain the hidden state tensor and utilize the hidden state tensor to update the knowledge graph for each entity to generate the changes in the risk classifications. The program code adds a dense layer to output the changes in the risk classifications generated by the long short-term memory layer.

Referring now to FIG. 7, a schematic of an example of a computing node, which can be a cloud computing node 10. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove. In FIG. 3, the convolutional LSTM network 330 can be executed and/or stored on a cloud computing node 10 (FIG. 7). In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 7, computer system/server 12 that can be utilized as cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random-access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs). Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter). Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user specific application configuration settings.

Platform as a Service (PaaS): the capability provided to the consumer is to deploy onto the cloud infrastructure consumer-created or acquired aF1pplications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

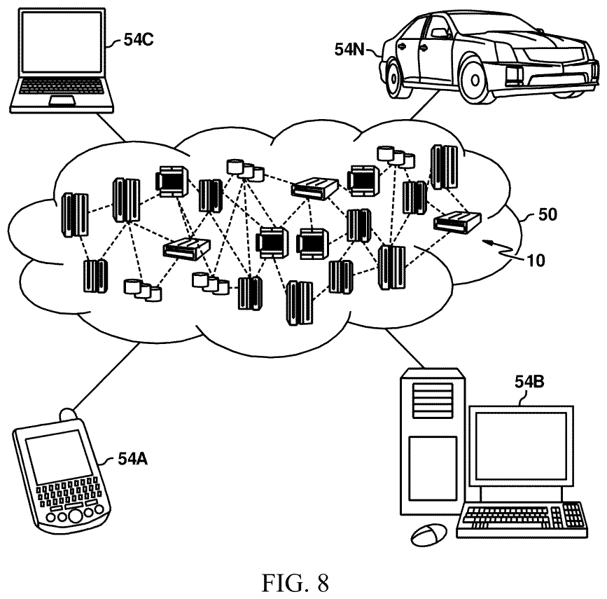

Referring now to FIG. 8, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 8 are intended to be illustrative only and that computing nodes 10 and cloud computing environment 50 can communicate with any type of computerized device over any type of network and/or network addressable connection (e.g., using a web browser).

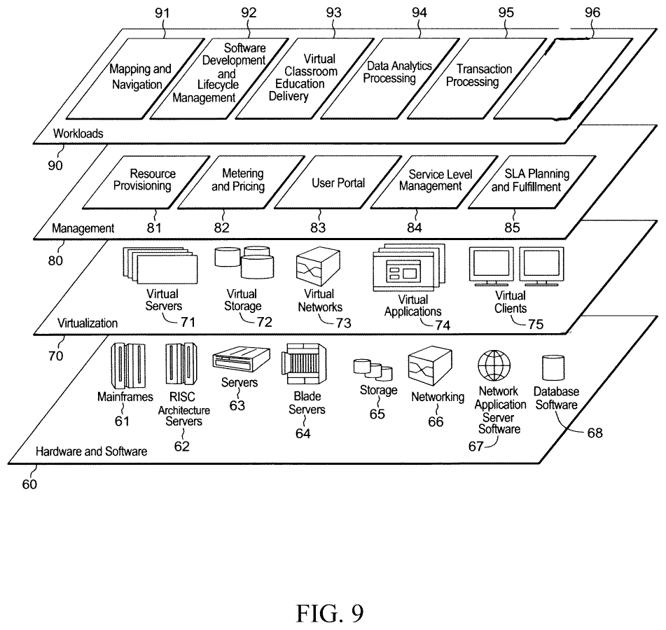

Referring now to FIG. 9, a set of functional abstraction layers provided by cloud computing environment 50 (FIG. 8) is shown. It should be understood in advance that the components, layers, and functions shown in FIG. 9 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75. Workloads can also include virtual examination centers or online examinations (not pictured).

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and identifying sources (human and location) for an infection and forecasting infection paths based on identifying these sources 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method, comprising:
obtaining, by one or more processors, various data relevant to one or more infected individuals, the various data comprising location data and infection data;
identifying, by the one or more processors, relationships and physical proximity between people comprising the one or more infected individuals and additional individuals, based on the various data;
generating, by the one or more processors, based on the various data, a geofence;
utilizing, by the one or more processors, the geofence, the relationships, the physical proximity, and the various data, to predict that a portion of the additional individuals are more likely than not to be infected with the infection;
generating, by the one or more processors, based on the relationships and physical proximity, a scoring model, wherein the scoring model assigns scores to entities comprising a group consisting of the one or more individuals, the portion of the additional individuals, and the physical locations to identify a source of the infection; and
applying, by the one or more processors, the scoring model to the group, to identify the source of the infection, wherein the source of the infection comprises at least one person from the group or at least one location from the physical locations.

2. The method of claim 1 further, wherein identifying the relationships and the physical proximity comprises:
utilizing, by the one or more processors, a Bayesian network.

3. The method of claim 1, wherein utilizing the geofence, the relationships, the physical proximity, and the various data, to predict the portion comprises:
determining, by the one or more processors, if an individual of the additional individuals is part of the portion by determining if the individual had a contact with at least one of the one or more infected individuals, wherein the contact is more likely than not to transmit the infection, based on at least a portion of the various data.

4. The computer-implemented of claim 1, wherein the various data further comprise location information for at least one infected individual from a social network or a surveillance network.

5. The computer-implemented method of claim 1, further comprising:
based on applying the scoring model, generating, by the one or more processors, a knowledge graph indicating a likelihood at least one person from the group entity is the source of the infection.

6. The computer-implemented method of claim 5, wherein the various data comprise geospatial data related to physical locations proximate to the one or more infected individuals, wherein the geospatial data comprise a geospatial map, the method further comprising:
generating, by the one or more processors, a heat map of infection risks based on superimposing, by the one or more processors, the knowledge graph on the geospatial map.

7. The computer-implemented method of claim 1, further comprising:
generating, by the one or more processors, based on applying the scoring model, a timeline of propagation for the infection.

8. The computer-implemented method of claim 6, further comprising:
providing, by the one or more processors, the knowledge graph to a neural network comprising a convolutional layer and a long short-term memory layer, wherein the convolutional layer encodes risk classifications based on a portion of the various data, and wherein the long short-term memory layer predicts changes in the risk classifications over time; and
updating, by the one or more processors, the knowledge graph based on the risk classifications.

9. The computer-implemented method of claim 1, further comprising:
based on applying the scoring model, generating, by the one or more processors, a knowledge graph indicating a likelihood at least one individual of the portion is the source of the infection;
monitoring, by the one or more processors, the at least one individual to determine actual results regarding whether the at least one individual is infected with the infection;
comparing, by the one or more processors, the actual results with the knowledge graph to identify discrepancies; and
updating, by the one or more processors, the scoring model to address the discrepancies.

10. The computer-implemented method of claim 1, further comprising:
comparing, by the one or more processors, results of applying the scoring model with a dataset selected from the group consisting of: an historic dataset of infection occurrence or a proxy dataset of infection occurrence;
based on the comparing, generating, by the one or more processors, an input sequence to train the scoring model;
providing, by the one or more processors, the input sequence to a neural network; and
updating, by the one or more processors, the scoring model based on output from the neural network generated from the input sequence.

11. The computer-implemented method of claim 8, wherein the generating the scoring model further comprises:
implementing, by the one or more processors, a neural network, the implementing comprising:
implementing, by the one or more processors, the convolutional layer comprising at least one convolutional block, wherein an encoding network of the convolutional layer comprises maximum pooling, wherein the convolutional layer compresses the knowledge graph for each entity into a hidden state tensor;
adding, by the one or more processors, the long short-term memory layer to obtain the hidden state tensor and utilize the hidden state tensor to update the knowledge graph for each entity to generate the changes in the risk classifications; and
adding, by the one or more processors, a dense layer to output the changes in the risk classifications generated by the long short-term memory layer.

12. A computer program product comprising:
a computer readable storage medium readable by one or more processors of a shared computing environment comprising a computing system and storing instructions for execution by the one or more processors for performing a method comprising:
obtaining, by the one or more processors, various data relevant to one or more infected individuals, the various data comprising location data and infection data;
identifying, by the one or more processors, relationships and physical proximity between people comprising the one or more infected individuals and additional individuals, based on the various data;
generating, by the one or more processors, based on the various data, a geofence;
utilizing, by the one or more processors, the geofence, the relationships, the physical proximity, and the various data, to predict that a portion of the additional individuals are more likely than not to be infected with the infection;
generating, by the one or more processors, based on the relationships and physical proximity, a scoring model, wherein the scoring model identifies a source of the infection; and
applying, by the one or more processors, the scoring model to a group consisting of the one or more individuals, the portion of the additional individuals, and the physical locations, to identify the source of the infection, wherein the source of the infection comprises at least one person from the group or at least one location from the physical locations.

13. The computer program product of claim 12, wherein identifying the relationships and the physical proximity comprises:
utilizing, by the one or more processors, a Bayesian network.

14. The computer program product of claim 12, wherein utilizing the geofence, the relationships, the physical proximity, and the various data, to predict the portion comprises:
determining, by the one or more processors, if an individual of the additional individuals is part of the portion by determining if the individual had a contact with at least one of the one or more infected individuals, wherein the contact is more likely than not to transmit the infection, based on at least a portion of the various data.

15. The computer program product of claim 12, wherein the various data further comprise location information for at least one infected individual from a social network or a surveillance network.

16. The computer program product of claim 12, further comprising:
based on applying the scoring model, generating, by the one or more processors, a knowledge graph indicating a likelihood at least one person from the group entity is the source of the infection.

17. The computer program product of claim 16, wherein the various data comprise geospatial data related to physical locations proximate to the one or more infected individuals, wherein the geospatial data comprise a geospatial map, the method further comprising:
generating, by the one or more processors, a heat map of infection risks based on superimposing, by the one or more processors, the knowledge graph on the geospatial map.

18. The computer program product of claim 12, the method further comprising:
generating, by the one or more processors, based on applying the scoring model, a timeline of propagation for the infection.

19. The computer program product of claim 17, the method further comprising:
providing, by the one or more processors, the knowledge graph to a neural network comprising a convolutional layer and a long short-term memory layer, wherein the convolutional layer encodes risk classifications based on a portion of the various data, and wherein the long short-term memory layer predicts changes in the risk classifications over time; and
updating, by the one or more processors, the knowledge graph based on the risk classifications.

20. A computer system comprising:
a memory;
the one or more processors in communication with the memory;
program instructions executable by the one or more processors to perform a method, the method comprising:
obtaining, by the one or more processors, various data relevant to one or more infected individuals, the various data comprising location data and infection data;
identifying, by the one or more processors, relationships and physical proximity between people comprising the one or more infected individuals and additional individuals, based on the various data;
generating, by the one or more processors, based on the various data, a geofence;
utilizing, by the one or more processors, the geofence, the relationships, the physical proximity, and the various data, to predict that a portion of the additional individuals are more likely than not to be infected with the infection;
generating, by the one or more processors, based on the relationships and physical proximity, a scoring model, wherein the scoring model identifies a source of the infection; and
applying, by the one or more processors, the scoring model to a group consisting of the one or more individuals, the portion of the additional individuals, and the physical locations, to identify the source of the infection, wherein the source of the infection comprises at least one person from the group or at least one location from the physical locations.
